(19) United States
(12) Patent Application Publication
(10) Pub. No.: US 2005/0214301 A1
(43) Pub. Date: Sep. 29, 2005

(54) Antibodies specific for BCR-ABL fusion protein and uses thereof

(75) Inventors: Randall K. Wetzel, Danvers, MA (US); Carrie A. Brown, Burlington, MA (US)

Correspondence Address: JAMES GREGORY CULLEM, ESQ.;INTELLECTUAL PROPERTY COUNSEL, CELL SIGNALING TECHNOLOGY, INC., 166B CUMMINGS CENTER, BEVERLY, MA, 01915, US

(73) Assignee: CELL SIGNALING TECHNOLOGY, INC.

(21) Appl. No.: 10/807,799

(22) Filed: Mar. 24, 2004

(51) Int. Cl. A61K 39/395; C12N 5/06; C07K 16/30

(52) U.S. Cl. 424/155.1; 530/388.8; 435/344

(57) ABSTRACT

The invention provides isolated antibodies that specifically bind human P210 BCR-ABL fusion protein, but do not bind the respective wild type BCR and c-ABL proteins. The detection of this fusion protein is relevant to CML and other diseases characterized by the P210 BCR-ABL translocation. Also provided are methods for determining the level or expression of P210 BCR-ABL in a biological sample, or identifying a compound that modulates such expression, by using the disclosed BCR-ABL specific antibodies.

### FIELD OF THE INVENTION

The invention relates generally to antibodies, and more particularly to antibodies against signal transduction proteins and their uses.

### BACKGROUND OF THE INVENTION

Many cancers are characterized by disruptions in cellular signaling pathways that lead to aberrant control of cellular processes, or to uncontrolled growth and proliferation of cells. These disruptions are often caused by changes in the phosphorylation state, and thus the activity of, particular signaling proteins. Among these cancers are hematopoietic diseases, such as chronic myelogenous leukemia (CML) and acute lymphocytic leukemia (ALL). There are about 4,700 new cases of CML in the United States annually, and it is estimated that 2,300 patients will die annually from the disease in the United States alone. See “Cancer Facts and Figures 2002,” American Cancer Society. There are about 3,500 new cases of ALL in the United States annually, and it is estimated that 1,400 patients will die annually from the disease in the United States alone. In children, leukemia is the most common type of cancer, and ALL is the most prevalent of these childhood leukemias. See id.

It has been directly demonstrated that the BCR-ABL oncoprotein, a protein tyrosine kinase, is the causative agent in human chronic myelogenous leukemia (CML). See Skorski et al., J. Clin Invest. 92:194-202 (1993); Snyder et al., Blood 82: 600-605 (1993). The BCR-ABL oncoprotein is generated by the translocation of gene sequences from the c-ABL protein tyrosine kinase on chromosome 9 into BCR sequences on chromosome 22, producing the so-called Philadelphia chromosome. See, e.g. Kurzock et al., N. Engl. J. Med. 319: 990-998 (1988); Rosenberg et al., Adv. in Virus Res. 35: 39-81 (1988). The BCR-ABL oncogene has been found in at least 90-95% of cases of CML. See, e.g. Fialkow et al., Am. J. Med. 63:125-130 (1977). The translocation is also observed in approximately 20% of adults with acute lymphocytic leukemia (ALL), 5% of children with ALL, and 2% of adults with acute myelogenous leukemia (AML). See, e.g. Whang-Peng et al., Blood 36:448-457 (1970); Look, Semin. Oncol. 12:92-104 (1985).

The BCR-ABL gene produces three alternative chimeric proteins, P230 BCR-ABL, P210 BCR-ABL, and P190 BCR-ABL. Of these, P210 BCR-ABL is characteristic of CML while P190 BCR-ABL is characteristic of ALL. BCR-ABL proteins exhibit heightened tyrosine kinase and transforming capabilities compared to the normal c-ABL protein. See, e.g. Konopka et al., Cell 37: 1035-1042 (1984). Many reports have indicated that BCR-ABL indeed acts as an oncogene and causes a variety of hematological malignancies, including granulocytic hyperplasia resembling human CML, myelomonocytic leukemia, ALL, lymphomas, and erythroid leukemia, in vivo. See, e.g. Lugo et al., MCB 9: 1263-1270 (1989); Daley et al., Science 247: 824-830 (1990); Honda, Blood 91: 2067-2075 (1998).

As a result, BCR-ABL has become a target for the development of therapeutics to treat leukemia. Most recently, Gleevec® (STI571), a small molecule inhibitor of the ABL kinase, has been approved for the treatment of CML. This drug is the first of a new class of anti-proliferative agents designed to interfere with the signaling pathways that drive the growth of tumor cells. The development of this drug represents a significant advance over the conventional therapies for CML and ALL, chemotherapy and radiation, which are plagued by well known side-effects and are often of limited effect since they fail to specifically target the underlying causes of the malignancies. However, Gleevec®, like many other therapeutics in development, only targets a single signaling protein among several implicated in the progression of the disease.

Clinical results since the introduction of Gleevec®have shown that patients often develop resistance to Gleevec®. See, e.g. Sawyers, Science 294(5548): 1834 (2001). The mechanism of resistance may vary from patient to patient, but is most often a result of mutations in the BCR-ABL DNA that results in a variant kinase that is not affected by the inhibitor. See, e.g., Mercedes, Science 294(5548): 1834 (2001). Resistance may also occur through increased expression of the BCR-ABL protein. See, e.g. Keeshan, Leukemia (12):1823-33 (2001). Improved BCR-ABL kinase inhibitors are now being developed that will target the mutant forms of BCR-ABL kinase. Patients may be switched to these inhibitors, or to increased doses of Gleevec®, depending upon the ability to detect restored BCR-ABL expression in patient samples. Accordingly, in order to most effectively treat CML patients, it will be crucial to develop a suitable assay for detecting BCR-ABL expression and kinase activity in patients undergoing Gleevec® treatment.

Presently, tumor burden or residual disease and BCR-ABL expression in CML patients is detected by genetic tests such as FISH. This test has the drawbacks of being unreliable for some labs and being expensive. In addition, the mRNA level in a sample may not correlate with the protein level or activity of BCR-ABL. Accordingly, the development of a simpler, less costly, antibody-based test for detecting BCR-ABL activity would be desirable. The BCR-ABL fusion protein may presently be detected by using antibodies against either the wild type BCR or c-ABL proteins. See, e.g., Wang and Arlinghaus, Cancer Research 51(11): 3048-51 (1991); U.S. Pat. No. 5,369,008 (Issued Nov. 29, 1994). This approach does not employ a single BCR-ABL fusion protein specific antibody. Similar approaches have been described employing multiple antibodies, each specific for a different epitope in either wild-type BCR and ABL, in an attempt to detect BCR-ABL fusion protein. See Berendes et al., U.S. Pat. No. 6,610,498 (Issued Aug. 26, 2003). Again, this approach does not employ a single antibody that is truly BCR-ABL fusion protein-specific, as the antibodies do not bind a unique epitope present only in the BCR-ABL fusion junction region. These methods are further limited by time-consuming biochemical techniques, such as Western blots, that must be employed to separate the signal of the BCR-ABL fusion protein itself from the signal of the wild type protein. Accordingly, the current reagents and methods are not well suited for clinical use.

More suitable would be development of an antibody-based assay capable of specifically detecting the BCR-ABL fusion protein itself. However, previous attempts to develop BCR-ABL fusion protein-specific antibodies have been unsuccessful. See, e.g., Falini and Mason, Blood 99: 409-426 (2002). Current immunocytochemical methods to detect genetic fusions rely on the differential sub-cellular localization or atypical expression pattern of the fusion protein compared to the wild type. This type of assay is limited, however, to only a very few translocations not including the BCR-ABL translocation. See Falini and Mason, supra.

Accordingly, there remains a need for the development of BCR-ABL fusion protein specific antibodies to enable new, simple, reliable, and clinically suitable assays for determining the relative percentage of tumor cells (those expressing BCR-ABL) to normal cells. Such antibodies would greatly assist in the staging of a CML patient's disease, which is particularly important for the treatment of CML, in which the detection of the transition from chronic phase to acute or blast crisis is critical for patient care. BCR-ABL fusion protein specific antibodies would also be highly useful for identifying disease cells for further signaling analysis.

Presently, CML cells are identified using flow cytometry through the use of a number of cell-surface markers, but this assay is not precise and may result in misidentifying normal cells as CML cells. Development of new antibodies that specifically detect the BCR-ABL fusion protein would enable more direct and reliable identification of CML cells, and would be well suited to the clinical analysis of BCR-ABL kinase activity using sensitive and widely-used techniques such as immuno-histochemistry (IHC) and flow cytometry (FC). Such new methods would greatly assist in optimally treating each CML patient as resistance to Gleevec® or other BCR-ABL targeted therapies develops.

### SUMMARY OF THE INVENTION

The invention provides novel antibodies that specifically detect the human P210 BCR-ABL fusion protein, which results from the Philadelphia chromosome translocation involving wild-type BCR and c-ABL. Also provided are methods for determining the protein expression level or activity of BCR-ABL in a biological sample, profiling BCR-ABL protein expression in a test tissue, and identifying a compound that modulates BCR-ABL expression or activity, by using a BCR-ABL fusion protein specific antibody. In some embodiments, the sample or test tissue is taken from a subject having or suspected of having a disease, such as CML, in which the BCR-ABL genetic translocation is implicated.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1—is the amino acid sequence (1-letter code) of the human BCR-ABL (SEQ ID NO: 1) fusion protein junction region. The peptide sequence encompassing the fusion joint, and corresponding to the peptide sequence used to generate a fusion protein specific antibody, is indicated in bold (residues 94-108) (see Example 1). The underlined portion of the peptide belongs to the ABL protein (amino acid #26-35 in the wild-type sequence). The italicized portion of the peptide belongs to the BCR protein (amino acid #897-901 in the wild-type sequence).

FIG. 2A—is a Western blot analysis of eight different cell lines demonstrating that an exemplary BCR-ABL fusion protein-specific monoclonal antibody (clone #4H3) of the invention (see Example 1) detects only the P210 BCR-ABL protein and not the BCR wild type protein or the c-ABL wild type protein.

FIG. 2B—is a Western blot analysis of LNCaP prostate cancer cells and K562 cells demonstrating that wild type c-ABL is detected by a c-ABL specific antibody, but not by a BCR-ABL fusion specific antibody.

FIG. 3A—is a flow cytometric analysis of six different cell lines with a BCR-ABL specific monoclonal antibody (clone #4H3) and a BCR wild type-specific antibody demonstrating that the BCR-ABL antibody detects the BCR-ABL fusion protein in the CML K562 cell line, but does not detect the BCR protein or P190 BCR-ABL protein expressed in the other five cell lines. BCR antibody binding is indicated in gray, BCR-ABL antibody binding is indicated in white, and black bars indicate ratio of BCR-ABL to BCR. The highest ration is in K562 cells, which express P210 BCR-ABL.

FIG. 3B—is a flow cytometric analysis, using a BCR-ABL fusion protein specific monoclonal antibody of the invention, of a normal patient blood sample (light) compared to a CML patient sample (dark) demonstrating the detection of the BCR-ABL protein in CML patient samples compared to a negative signal in normal patient samples.

FIG. 4—is an immunohistochemical analysis of paraffin-embedded cells lines demonstrating BCR-ABL fusion protein specific monoclonal antibody correctly identifies the CML cell line (K562) and is negative in the T cell line (Jurkat) and the B cell line (Ramos), both of which lack the BCR-ABL translocation (Philadelphia chromosome).

### DETAILED DESCRIPTION OF THE INVENTION

The present invention provides novel antibodies that specifically bind the human P210 BCR-ABL fusion protein. This protein, a tyrosine kinase generated by the translocation of gene sequences from the c-ABL protein tyrosine kinase on chromosome 9 into BCR sequences on chromosome 22 (producing the so-called Philadelphia chromosome), has been directly demonstrated to be the causative agent in human chronic myelogenous leukemia (CML). See, e.g., Skorski et al., supra. The BCR-ABL oncogene has been found in at least 90-95% of cases of CML, and has also been observed in approximately 20% of adults with acute lymphocytic leukemia (ALL), 5% of children with ALL, and 2% of adults with acute myelogenous leukemia (AML). See, e.g. Fialkow et al., supra. The P210 chimeric form of BCR-ABL is associated with CML. The production of a P210 BCR-ABL fusion protein-specific antibody accordingly to the present invention is surprising, since previous attempts to generate such an antibody have failed. See, e.g. Falini et al., supra.

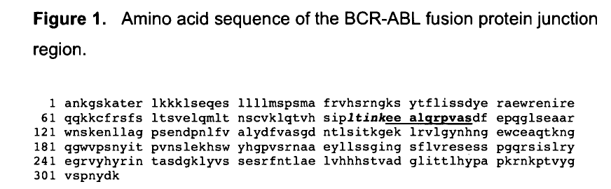

An exemplary P210 BCR-ABL fusion protein specific antibody as disclosed herein was produced using a synthetic peptide antigen corresponding to the amino acid sequence of the BCR and c-ABL fusion joint (residues 94-108 of SEQ ID NO: 1) in the BCR-ABL fusion protein junction region (see FIG. 1). See also Example 1. This antibody specifically binds the fusion junction that is present in BCR-ABL but not wild-type BCR or c-ABL proteins.

Further provided by the present invention are novel and powerful new methods of using a BCR-ABL specific antibody to identify patient samples that contain disease cells implicating the BCR-ABL translocation, for example, CML patients. Detection of BCR-ABL in a biological sample may be carried out in a variety of clinically-suitable assay formats, such as flow cytometry or by immunocytochemistry. Antibodies of the invention also enable new methods for the quantitative analysis of BCR-ABL expression levels, which may be translated into a measurement of tumor burden or drug effectiveness, for example, effectiveness of Gleevec® therapy in a CML patient.

The further aspects, advantages, and embodiments of the invention are described in more detail below. All references cited herein are hereby incorporated by reference.

A. Antibodies and Cell Lines

As used herein, the term “BCR-ABL antibody” is used interchangeably with “BCR-ABL specific antibody,” “BCR-ABL fusion protein specific antibody” and/or “BCR-ABL fusion protein junction antibody” and means an isolated antibody or antibodies that specifically bind(s) the human P210 BCR-ABL fusion protein junction (see FIG. 1; SEQ ID NO: 1) expressed as a result of the BCR and c-ABL genetic translocation in the Philadelphia chromosome, but does not bind wild type BCR or c-ABL. The term “does not bind” with respect to disclosed antibodies means does not substantially react with as compared to binding to the fusion protein junction. The term “BCR-ABL fusion protein” means a BCR-ABL protein or polypeptide comprising the fusion protein junction or splice site.

BCR-ABL fusion protein-specific antibodies of the invention specifically bind to human P210 BCR-ABL and do not substantially bind to wild type BCR or c-ABL. BCR-ABL fusion specific antibodies also bind to highly homologous and equivalent BCR-ABL fusion junctions in other species, for example murine or rabbit BCR-ABL. The BCR-ABL antibodies of the invention include (a) monoclonal antibodies that specifically bind the BCR-ABL fusion protein at the junction region, (b) purified polyclonal antibodies that specifically bind to the BCR-ABL fusion protein junction, (c) antibodies (monoclonal or polyclonal) that specifically bind to the fusion protein junction (or more preferably the epitope) bound by the exemplary BCR-ABL monoclonal antibody disclosed in the Examples herein, (d) antibodies as described in (a)-(c) above that bind equivalent and highly homologous BCR-ABL fusion sites in other non-human species (e.g. mouse, rat), as disclosed herein, and (e) fragments of (a)-(d) above that bind to the antigen (or more preferably the epitope) bound by the exemplary antibodies disclosed herein.

Such antibodies and antibody fragments that are within the scope of the present invention may be produced by a variety of techniques well known in the art, as further discussed below. Antibodies that bind to the fusion junction (and/or fusion joint) of the BCR-ABL protein that is bound by the exemplary BCR-ABL antibody of the Examples herein can be identified in accordance with known techniques.

The preferred epitopic site of a BCR-ABL fusion protein specific antibody of the invention is a peptide fragment consisting essentially of about 11 to 17 amino acids including the fusion joint (or splice site) of the human BCR-ABL fusion protein region (see FIG. 1), wherein about 5 to 9 amino acids are positioned on each side of the fusion joint that occurs at residues 98/99 in the fusion region (for example, residues 94-108 of SEQ ID NO: 1). It will be appreciated antibodies that specifically bind shorter or longer peptides comprising the BCR-ABL splice junction itself are within the scope of the present invention. In one preferred embodiment, there is provided a BCR-ABL fusion protein junction specific antibody that specifically binds a peptide comprising residues 94-108 of human BCR-ABL (see FIG. 1; SEQ ID NO: 1). In another preferred embodiment, there is provided a BCR-ABL fusion protein junction specific antibody that specifically binds a peptide comprising residues 97-101 of human BCR-ABL (see FIG. 1; SEQ ID NO: 1). The amino acid sequence of human wild type BCR (see accession P00519) and c-ABL (see accession #P11274) are both known and published, as are the sequences of BCR and c-ABL from other species.

The invention is not limited to BCR-ABL antibodies, but includes equivalent molecules, such as protein binding domains or nucleic acid aptamers, which bind, in a fusion-protein specific manner, to essentially the same epitope to which the BCR-ABL antibody of the invention bind. See, e.g., Neuberger et al., Nature 312:604 (1984). Such equivalent non-antibody reagents may be suitably employed in the methods of the invention further described below. The invention also includes fusion junction specific antibodies that bind to mutant forms of human BCR-ABL that contain one or more mutations in or near the fusion junction region (see FIG. 1). Such mutant forms can readily be identified by comparing the fusion junction region of the mutant BCR-ABL with the normal BCR-ABL fusion junction region (see FIG. 1; SEQ ID NO: 1) to identify specific residues at which mutations exist.

The term “antibody” or “antibodies” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed specific antibodies made according to the method disclosed in U.S. Pat. No. 4,676,980 (Segel et al.)

Polyclonal antibodies of the invention may be produced according to standard techniques by immunizing a suitable animal (e.g., rabbit, goat, etc.) with an antigen encompassing the fusion joint of the translocated BCR and ABL genes, collecting immune serum from the animal, and separating the polyclonal antibodies from the immune serum, and purifying polyclonal antibodies having the desired specificity, in accordance with known procedures. In a preferred embodiment, the antigen is a synthetic peptide antigen comprising the BCR-ABL sequence surrounding and including the fusion joint, as described above, the antigen being selected and constructed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85:21-49 (1962)).

A particularly preferred synthetic peptide antigen useful for producing BCR-ABL antibodies of the invention consists of residues 94-108 in the human BRC-ABL fusion junction, as described in Example 1, below. It will be appreciated by those of skill in the art that longer or shorter peptide antigens may be employed. See Id. For example, a longer peptide comprising residues 94-108 may be employed as an antigen. Similarly, a shorter polypeptide comprising the BCR-ABL splice site and 2 or more residues flanking each side of it may suitably be employed as a peptide antigen. Similarly, antigens may be prepared that correspond to mutant forms of human BCR-ABL fusion junction (identified by comparing the mutant fusion junction sequence with the BCR-ABL sequence identified in FIG. 1). Polyclonal BCR-ABL antibodies produced as described herein may be screened and isolated as further described below.

Monoclonal antibodies of the invention may be produced in a hybridoma cell line according to the well-known technique of Kohler and Milstein. Nature 265:495-97 (1975); Kohler and Milstein, Eur. J. Immunol. 6:511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse and, after a sufficient time (in keeping with conventional techniques), the mouse sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Alternatively, immortalized monoclonal antibody producing cell lines may be produced without fusion hybridomas, for example, by using transgenic spleen cells that are conditionally immortal. See, e.g. Pasqualini et al., PNAS 101(1): 257-259 (2004); Jat et al., U.S. Pat. No. 5,866,759 (Issued Feb. 2, 1999).

Monoclonal Fab fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

The invention also provides immortalized cell lines, such as hybridoma clones, constructed as described above, that produce BCR-ABL monoclonal antibodies of the invention. Similarly, the invention includes recombinant cells producing a BCR-ABL antibody as disclosed herein, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.) Hybridomas and BCR-ABL fusion junction specific antibodies provided by the invention include hybridoma clone #4H3, deposited on Mar. 9, 2004, ATCC Accession No. PTA-5851.

BCR-ABL antibodies of the invention, whether polyclonal or monoclonal, may be screened for epitope and fusion protein junction specificity according to standard techniques. See, e.g. Czernik et al., Methods in Enzymology, 201: 264-283 (1991). For example, the antibodies may be screened against a peptide library by ELISA to ensure specificity for both the desired antigen and for reactivity only with the fusion form of the antigen. The antibodies may also be tested by Western blotting against cell preparations containing BCR-ABL, BCR and c-ABL, e.g. cell lines expressing BCR-ABL, BCR and c-ABL, to confirm reactivity with the desired fusion target. BCR-ABL antibodies of the invention may exhibit some limited cross-reactivity with non-BCR-ABL epitopes. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-BCR-ABL proteins is readily characterized by Western blotting alongside markers of known molecular weight. Amino acid sequences of cross-reacting proteins may be examined to identify sites highly homologous to the BCR-ABL sequence surrounding the fusion site.

BCR-ABL antibodies may be further characterized by flow cytometry using normal and Philadelphia chromosome-positive CML patient samples (blood or marrow). Flow cytometry may be carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary BCR-ABL fusion protein specific antibody, washed and labeled with a fluorescent-labeled secondary antibody. Additional fluorochrome-conjugated marker antibodies (e.g. CD45, CD34) may also be added at this time to aid in the subsequent identification of specific hematopoietic cell types. The cells would then be analyzed on a flow cytometer (e.g. a Beckman Coulter FC500) according to the specific protocols of the instrument used. Such an analysis would identify the presence of the BCR-ABL fusion protein in CML cells.

BCR-ABL fusion protein specific antibodies of the invention may also be advantageously conjugated to fluorescent dyes (e.g. Alexa488, PE) for use in multi-parametric analyses along with other signal transduction (phospho-CrkL, phospho-Erk 1/2) and/or cell marker (CD34) antibodies.

BCR-ABL antibodies may be further characterized via immunohistochemical (IHC) staining using normal and diseased tissues to examine BCR-ABL expression in diseased tissue. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

BCR-ABL antibodies of the invention specifically bind to the fusion junction of human BCR-ABL protein (which is expressed as a result of the Philadelphia genetic translocation), but are not limited only to binding the human species, per se. The invention includes fusion-specific antibodies that also bind conserved and highly-homologous BCR-ABL fusion junctions/sites in other species (e.g. mouse, rat, monkey, yeast), in addition to binding the human BCR-ABL fusion site, are within the scope of the present invention. Additional highly-homologous sites conserved in other species, which are in within the scope of the invention, can readily be identified by standard sequence comparisons, such as using BLAST, with the human BCR-ABL sites disclosed herein.

B. Detection & Profiling Methods

BCR-ABL fusion protein specific antibodies provided by the invention enable powerful and previously unavailable immunological methods for the rapid and simple detection of BCR-ABL expression and/or activity in a biological sample, for example, a CML patient sample. The methods disclosed herein may be employed with any biological sample potentially containing, or suspected of containing, fused BCR and c-ABL proteins. Biological samples taken from human subjects for use in the methods disclosed herein are generally biological fluids such as serum, blood plasma, fine needle aspirant, ductal lavage, bone marrow sample or ascites fluid. In the alternative, the sample taken from the subject can be a tissue sample (e.g., a biopsy tissue), such as bone marrow or tumor tissue, or a cell lysate, whether or not purified.

In one embodiment, the invention provides a method for detecting BCR-ABL fusion protein in a biological sample by (a) contacting a biological sample potentially (or suspected of) containing a BCR-ABL fusion polypeptide with at least one BCR-ABL fusion specific antibody, under conditions suitable for formation of an antibody-antigen complex, and (b) detecting the presence of the complex in the sample, wherein the presence of the complex indicates the presence of the BCR-ABL fusion polypeptide in the sample.

In certain preferred embodiments, the biological sample has been contacted with at least one BCR-ABL inhibitor or is obtained from a subject treated with such inhibitor. Accordingly, changes in BCR-ABL expression resulting from contacting a biological sample with a test compound, such as a BCR-ABL inhibitor, may be examined to determine effect of such compound. Exemplary inhibitors of BCR-ABL include, but are not limited to Gleevec® (STI-571), and its analogues. Inhibitory compounds may be targeted inhibitors that modulate post-kinase activity of BCR-ABL, or may be upstream expression inhibitors, such as siRNA or anti-sense inhibitors. In another preferred embodiment, the compound is being tested for inhibition of BCR-ABL activity or expression. Such compound may, for example, directly inhibit BCR-ABL activity, or may indirectly inhibit its activity by, e.g., inhibiting another kinase that phosphorylates and thus activates BCR-ABL.

Biological samples may be obtained from a subject at risk of, potentially, or suspected of, having a disease or condition involving altered BCR-ABL expression or activity (e.g., CML, ALL). For example, samples may be analyzed to monitor subjects who have been previously diagnosed as having CML, to screen subjects who have not been previously diagnosed as having CML, or to monitor the desirability or efficacy of therapeutics targeted at BCR-ABL. In the case of CML, for example, the subjects will most frequently be adult patients undergoing Gleevec® treatment and are at risk for the development of Gleevec® resistance.

In another embodiment, the invention provides a method for profiling BCR-ABL expression in a test tissue potentially having (or suspected of involving) altered BCR-ABL translocation, by (a) contacting the test tissue with at least one BCR-ABL fusion specific antibody under conditions suitable for formation of an antibody-antigen complex, (b) detecting the presence of the complex in the test tissue, wherein the presence of the complex indicates the presence of BCR-ABL fusion protein in the test tissue, and (c) comparing the presence of BCR-ABL detected in step (b) with the presence of BCR-ABL in a control tissue, wherein a difference in BCR-ABL expression profiles between the test and control tissues indicates the presence of a genetic translocation the test tissue. In some preferred embodiments, the test tissue is bone marrow or blood samples potentially having (or suspected of involving) BCR-ABL fusion protein expression.

The methods described above are applicable to examining tissues or samples from any disease or condition involving or characterized by altered BCR-ABL expression, particularly CML, in which expression of BCR-ABL has predictive value as to the outcome of the disease or the response of the disease to therapy. It is anticipated that the BCR-ABL antibodies will have diagnostic utility in a disease characterized by, or involving, altered BCR-ABL expression. The methods are applicable, for example, where samples are taken from a subject has not been previously diagnosed as having CML, nor has yet undergone treatment for CML, and the method is employed to help diagnose the disease, or monitor the possible progression of the condition, or assess risk of the subject developing a disease involving BCR-ABL.

Such diagnostic assay may be carried out prior to preliminary blood evaluation or surgical surveillance procedures. Such a diagnostic assay may be advantageously employed to identify patients with BCR-ABL fusion protein expression who would be most likely to respond to therapeutics targeted at inhibiting BCR-ABL activity, such as Gleevec® or its analogues. Such a selection of patients would be useful in the clinical evaluation of efficacy of future BCR-ABL-targeted therapeutics as well as in the future prescription of such drugs to patients. Alternatively, the methods are applicable where a subject has been previously diagnosed as having a disease involving altered BCR-ABL signaling, such as CML, and possibly has already undergone treatment for the disease, and the method is employed to monitor the progression of the disease involving BCR-ABL expression, or the treatment thereof.

In another embodiment, the invention provides a method for identifying a compound which modulates expression of BCR-ABL in a test tissue, by (a) contacting the test tissue with the compound, (b) detecting the level of BCR-ABL polypeptide in said the test tissue of step (a) using at least one BCR-ABL fusion protein specific antibody under conditions suitable for formation of an antibody-antigen complex, and (c) comparing the level of fused BCR-ABL detected in step (b) with the presence of fused BCR-ABL in a control tissue not contacted with the compound, wherein a difference in BCR-ABL levels between the test and control tissues identifies the compound as a modulator of BCR-ABL activity and expression. In some preferred embodiments, the test tissue is a taken from a subject potentially (or suspected of) having CML and the compound is a BCR-ABL inhibitor. The compound may modulate BCR-ABL levels negatively, for example by decreasing the kinase activity of BCR-ABL. Alternatively, BCR-ABL protein levels may be monitored to determine the efficacy of a compound targeted at any kinase downstream of BCR-ABL such as ERK1/2.

Conditions suitable for the formation of antibody-antigen complexes or reagent-BCR-ABL complexes are well known in the art (see part (d) below and references cited therein). It will be understood that more than one antibody may be used in the practice of the above-described methods. For example, a BCR-ABL antibody and a phospho-specific antibody to another kinase may be simultaneously employed to detect BCR-ABL expression as well as activity in one step.

C. Immunoassay Formats & Diagnostic Kits

Assays carried out in accordance with methods of the present invention may be homogeneous assays or heterogeneous assays. In a homogeneous assay the immunological reaction usually involves a BCR-ABL-specific reagent (e.g. a BCR-ABL antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a BCR-ABL-specific reagent (e.g., the BCR-ABL antibody of the invention), and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof, which may be useful for carrying out the methods disclosed herein, are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No.4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of BCR-ABL is detectable compared to background.

BCR-ABL antibodies disclosed herein may be conjugated to a solid support suitable for a diagnostic assay (e.g., beads, plates, slides or wells formed from materials such as latex or polystyrene) in accordance with known techniques, such as precipitation. Antibodies of the invention, or other BCR-ABL binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

BCR-ABL antibodies of the invention are advantageously employed in a flow cytometry assay to determine the expression status of BCR-ABL in patients before, during, and after treatment with a drug targeted at inhibiting BCR-ABL kinase activity. For example, bone marrow cells or peripheral blood cells or smears from patients may be analyzed by flow cytometry for BCR-ABL expression, as well as for markers identifying various hematopoietic cell types. In this manner, BCR-ABL expression status of the diseased cells may be specifically characterized, using this clinically suitable assay format. Flow cytometry may be carried out according to standard methods. See, e.g. Chow et al., Cytometry (Communications in Clinical Cytometry) 46:72-78 (2001). Briefly and by way of example, the following protocol for cytometric analysis may be employed: fixation of the cells with 2% paraformaldehyde for 10 minutes at 37° C. followed by permeabilization in 90% methanol for 30 minutes on ice. Cells may then be stained with the primary BCR-ABL antibody, washed and labeled with a fluorescent-labeled secondary antibody. The cells would then be analyzed on a flow cytometer (e.g. a Beckman Coulter FC500) according to the specific protocols of the instrument used. Such an analysis would identify the presence of the BCR-ABL fusion protein in a cell of interest and reveal the drug response on the targeted BCR-ABL kinase.

Diagnostic kits for carrying out the methods disclosed above are also provided by the invention. Such kits comprise at least one BCR-ABL fusion protein specific antibody of the invention. In one embodiment, the invention provides a kit for the detection of BCR-ABL in a biological sample comprising (a) at least one BCR-ABL antibody of the invention (i.e. a fusion protein-specific antibody that binds BCR-ABL) and (b) at least one secondary antibody conjugated to a detectable group. The reagents may also include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

### Production of a BCR-ABL Fusion Protein-Specific Monoclonal Antibody

A 14 amino acid peptide antigen corresponding to residues spanning the splice region of human P210 BCR-ABL (see residues 94-108 in SEQ ID NO: 1), was constructed according to standard synthesis techniques using a Rainin/Protein Technologies, Inc., Symphony peptide synthesizer. See ANTIBODIES: A LABORATORY MANUAL, supra.; Merrifield, supra. This sequence includes the BCR-ABL splice site at residues 98/99, and comprises residues 94-98 (corresponding to BCR wild type residues 897-901) left of the splice site and residues 99-108 (corresponding to c-ABL wild type residues 26-35) right of the splice site. This peptide was coupled to KLH, and BALB/C mice were injected intraperatoneally (IP) with the antigen in complete Freunds adjuvant (500 g antigen per mouse). The mice were boosted with same antigen in incomplete Freund adjuvant (250 g antigen per mouse) every three weeks. After the fifth boost and a further pre-fusion boost, spleens were collected as needed for fusions. BCR-ABL specific monoclonal antibodies were produced from spleen cells of the immunized BALB/c mice following standard procedures (Harlow and Lane, 1988).

Briefly, the mouse spleen was fused to Ag8 mouse myeloma fusion partner cells according to the protocol of Kohler and Milstein (1975). Colonies originating from the fusion were first screened by ELISA for reactivity to the antigen peptide. 147 ELISA positive clones were identified in the first screen. Of these, only 49 clones were ELISA positive in the second screen. These clones were then tested by flow cytometry and by Western blot analysis against K562 cells. A number of clones positive on both flow cytometry and Western blot analysis were identified, of which, clone #4H3 was selected as the best. This clone was then subcloned by limited dilution. Mouse ascites was produced from the single clone obtained from subcloning.

Ascites fluid from clones obtained from the BCR-ABL fusion were further tested by flow cytometric analysis. The ascites fluid gave similar results on flow cytometry and Western blot analysis as observed with the cell culture supernatant, indicating specificity on BCR-ABL expressing K562 leukemia cells and lack of reactivity on cell lines expressing only the wild type BCR or c-ABL proteins or the P190 BCR-ABL fusion protein.

### EXAMPLE 2

### Western Blot Analysis Using a BCR-ABL Monoclonal Antibody

The BRC-ABL fusion protein-specific monoclonal antibody (see Example 1) was tested for specificity to the P210 BCR-ABL fusion protein using a Western blot assay. K562, 3T3, Jurkat, HeLa, SUPB15 and 3T3-ABL cell lines were cultured in DMEM or RPMI supplemented with 10% FBS. Of these cell lines, only the K562 cell line has the P210 BCR-ABL translocation. The SUPB15 cell line expresses the P190 BCR-ABL protein. For Western blot analysis, cells were collected, washed with PBS and directly lysed in either cell lysis buffer or denaturing urea buffer. The protein concentration of the cell lysates were measured. The loading buffer was added into cell lysate and the mixture was boiled at 100° C. for 5 minutes. The 15 μl (˜10 μg protein) of sample was added onto 6% SDS-PAGE gel. The standard Western blot was performed according to the Immunoblotting Protocol set out in the Cell Signaling Technology 2002 Catalog and Technical Reference, p. 282. The lysates were also probed with an antibody specific for BCR.

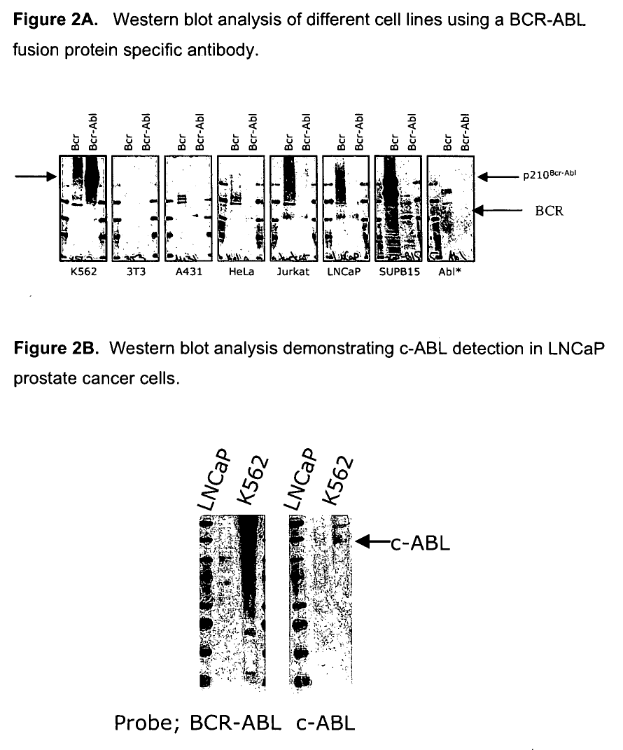

The results of the blots are shown in FIGS. 2A-2B. As shown in the Figures, the fusion-specific antibody, as expected, only recognizes the ˜210 kDa BCR-ABL protein. It does not recognize the c-ABL, BCR or P190 BCR-ABL proteins. The BCR protein is detected by the BCR antibody in all but the 3T3 lysates. The BCR-ABL antibody does not recognize a band at the same size as the BCR protein indicating that it does not cross-react with wild type BCR.

### EXAMPLE 3

### Flow Cytometric Analysis of BCR-ABL Expression Using a BCR-ABL Specific Antibody

BCR-ABL fusion protein specific monoclonal antibody #4H3 was used in flow cytometry to detect the P210 BCR-ABL protein in k562, Jurkat, Ramos, HeLa, SUPB15 and 3T3 cell lines. Of these cell lines, only the K562 cell line has the P210 BCR-ABL translocation (the SUPB15 cell lines expresses the P190 BCR-ABL protein). Following cell culture as described above, the cells were fixed with 2% paraformaldehyde for 10 minutes at 37° C. followed by cell permeabilization 90% with methanol for 30 minutes on ice. The fixed cells were then stained with the BCR-ABL primary antibody and a BCR primary antibody for 30 minutes at room temperature. The cells were then washed and stained with a FITC-labeled secondary antibody for 30 minutes at room temperature. The cells were then analyzed on a Beckman Coulter FC500 flow cytometer.

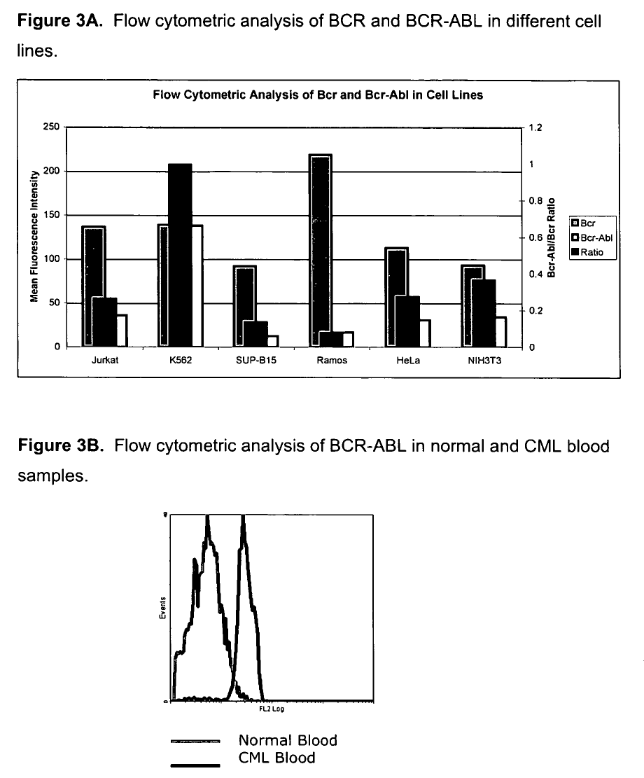

The results of the analysis are presented in FIG. 3A; the white bars represent the BCR-ABL staining intensity, the gray bars represent the BCR staining intensity and the black bars represent the ratio between the two intensities. The cytometric results matched the Western results described above; the only cell line that showed strong staining with the BCR-ABL antibody (high blue bar) is the K562 cell line. All the cell lines expressed wild type BCR as shown by the green bars. These results further demonstrating the specificity of the BCR-ABL antibody for the P210 BCR-ABL protein and not the BCR wild type protein or the P190 BCR-ABL protein.

To demonstrate the utility of a flow cytometric assay using the BCR-ABL antibody, patient blood samples were analyzed. Samples from one healthy patient and one patient with CML were analyzed using the BCR-ABL antibody as described above. The results of the analysis are presented in FIG. 3B. The light plot from the healthy patient shows minimal staining while the dark plot from the CML patient correctly confirms expression of the BCR-ABL fusion protein.

### EXAMPLE 4

### Immunohistochemical Analysis of BCR-ABL Expression Using a BCR-ABL Fusion Specific Antibody

For the IHC analysis, human K562, Jurkat and Ramos cell lines were obtained from the ATCC, Manassas, Va. and cultured as described above. The Jurkat and Ramos cell lines are leukemias that lack the BCR-ABL translocation while the K562 cell line has the translocation. Following cell culture, the cells were washed, spun down and cell pellets were fixed and embedded in paraffin. For IHC staining, 4 micron thick slices were cut from the paraffin blocks using a microtome and placed on glass slides. The sections may then be de-paraffinized with xylene and ethanol. The tissues were then microwaved for 10 minutes in a citrate pH 6.0 buffer for antigen retrieval. After a 10 minute incubation in 3% H₂O₂, the sections were blocked in 5% goat serum for 1 hour. The cell slides were then stained with the BCR-ABL antibody for overnight at 4° C. After 3 washes in TBS-Tween, the slides were then probed with a secondary antibody labeled with biotin. The slides are further developed with a avidin-biotin-HRP reagent (ABC kit) following standard manufacturer procedures. The slides were developed using a HRP substrate, NovaRed™, and counterstained with hematoxylin.

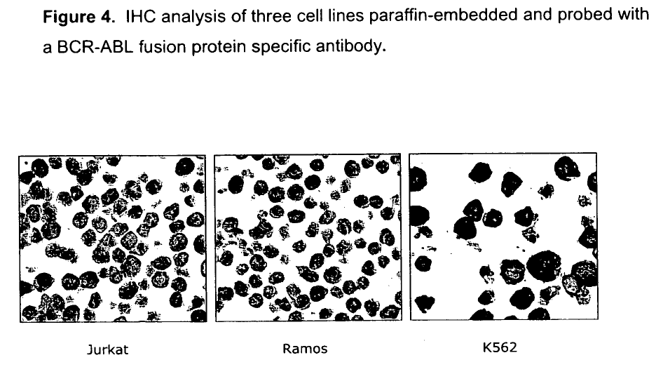

The results of the analysis are presented in FIG. 4. The strong staining observed in the K562 cells and the negative staining in the other two cell lines demonstrate the specificity and utility of a BCR-ABL fusion-specific antibody in determining P210 BCR-ABL expression using clinically-suitable methods, such as flow cytometry or IHC techniques.

1. An isolated antibody that specifically binds to human P210 BCR-ABL fusion protein (SEQ ID NO: 1), but does not bind wild type BCR or wild type c-ABL.

2. The antibody of claim 1, wherein said antibody binds a P210 BCR-ABL polypeptide comprising fusion joint residues 94 to 108 of SEQ ID NO: 1.

3. The antibody of claim 1, wherein said antibody binds a P210 BCR-ABL polypeptide comprising fusion joint residues 97 to 101 of SEQ ID NO: 1.

4. The antibody of claim 1, wherein said antibody is suitable for specifically detecting P210 BCR-ABL fusion protein in a cell-based assay selected from the group consisting of flow cytometry (FC), immunohistochemistry (IHC), or immunofluorescence (IF).

5. The antibody of claim 1, wherein said antibody is monoclonal.

6. An immortalized cell line producing the antibody of claim 5.

7. The cell line of claim 6, wherein said cell line is a hybridoma.

8. The cell line of claim 7, wherein said hybridoma is ATCC Accession No. PTA-5851.

9. A method for detecting the presence of P210 BCR-ABL fusion protein in a biological sample, said method comprising the steps of:
(a) contacting a biological sample potentially, or suspected of, containing P210 BCR-ABL fusion protein with at least one antibody of claim 1, under conditions suitable for formation of an antibody-BCR-ABL fusion protein complex; and
(b) detecting the presence of said complex in said biological sample, wherein the presence of said complex indicates the presence of P210 BCR-ABL fusion protein in said sample.

10. The method of claim 9, wherein said biological sample is obtained from a subject at risk of, or suspected of, having a disease involving BCR-ABL fusion protein expression.

11. The method of claim 10, wherein said disease is chronic myelogenous leukemia (CML).

12. The method of claim 9, wherein said biological sample has been contacted with at least one BCR-ABL inhibitor, or is obtained from a subject treated with such inhibitor.

13. The method of claim 9, wherein said biological sample has been contacted with a compound being tested for inhibition of BCR-ABL activity or expression.

14. A method for identifying a compound that modulates expression of P210 BCR-ABL fusion protein in a biological sample, said method comprising the steps of:
(a) contacting a test biological sample with a test compound,
(b) detecting the level of P210 BCR-ABL fusion protein in said test biological sample of step (a) using at least one antibody of claim 1 under conditions suitable for formation of an antibody-BCR-ABL fusion protein complex, and
(c) comparing the level of P210 BCR-ABL fusion protein detected in step (b) with the presence of P210 BCR-ABL fusion protein in a control sample not contacted with said test compound, wherein a difference in P210 BCR-ABL fusion protein levels in said test and control samples identifies said compound as a compound that modulates expression of P210 BCR-ABL fusion protein.

15. A kit for the detection of P210 BCR-ABL fusion protein in a biological sample, said kit comprising at least one detectable antibody of claim 1.


SEQUENCE LISTING (1 sequence)

SEQ ID NO: 1 - protein, 307 aa, Homo sapiens
  MISC_FEATURE (98)..(99): BCR-ABL fusion joint occurs a Lys-Glu, residues 98-99
ANKGSKATERLKKKLSEQESLLLLMSPSMAFRVHSRNGKSYTFLISSDYERAEWRENIREQQKKCFRSFSLTSVELQMLTNSCVKLQTVHSIPLTINKEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDK